(19) United States
(12) Patent Application Publication
(10) Pub. No.: US 2021/0235980 A1
(43) Pub. Date: Aug. 5, 2021

(54) MEDICAL-USE IMAGE PROCESSING DEVICE, ENDOSCOPE SYSTEM, AND MEDICAL-USE IMAGE PROCESSING METHOD

(71) Applicant: FUJIFILM Corporation, Tokyo (JP)

(72) Inventor: Masaaki OOSAKE, Kanagawa (JP)

(73) Assignee: FUJIFILM Corporation, Tokyo (JP)

(21) Appl. No.: 17/216,920

(22) Filed: Mar. 30, 2021

Related U.S. Application Data
(63) Continuation of application No. PCT/JP2019/038765, filed on Oct. 1, 2019.

(30) Foreign Application Priority Data
Oct. 12, 2018 (JP) 2018-193628

(51) Int. Cl. A61B 1/06; A61B 1/00; G06T 7/00; G06K 9/32; G06N 3/08; G06K 9/62

(52) U.S. Cl. CPC A61B 1/0669; A61B 1/00009; G06T 7/0012; A61B 1/00045; G06T 2207/10068; G06N 3/08; A61B 1/0638; G06K 9/627; G06K 9/3233

(57) ABSTRACT

A medical-use image processing device including: an image acquisition unit that acquires a medical-use image; a determination unit that determines an illumination mode at the time when the medical-use image is captured; a recognition unit that performs a first recognition for the medical-use image in a case where determination is made that the illumination mode is a first illumination mode, and performs a second recognition for the medical-use image in a case where determination is made that the illumination mode is a second illumination mode; and a display control unit that causes a display device to display a first display according to a result of the first recognition in a case where the determination is made that the illumination mode is the first illumination mode, and causes the display device to display a second display according to a result of the second recognition in a case where the determination is made that the illumination mode is the second illumination mode.

### CROSS-REFERENCE TO RELATED APPLICATIONS

This application is a Continuation of PCT International Application No. PCT/JP2019/038765 filed on Oct. 1, 2019, which claims priority under 35 U.S.C. § 119(a) to Japanese Patent Application No. 2018-193628 filed on Oct. 12, 2018. Each of the above application(s) is hereby expressly incorporated by reference, in its entirety, into the present application.

### BACKGROUND OF THE INVENTION

### 1. Field of the Invention

The present invention relates to a medical-use image processing device, an endoscope system, and a medical-use image processing method, and particularly to a medical-use image processing device, an endoscope system, and a medical-use image processing method for handling images captured in a plurality of illumination modes.

### 2. Description of the Related Art

10002 In the medical field, images of test objects captured with medical equipment are used for diagnosis, treatment, and the like, but “what kind of structure of a subject is clearly (or unclearly) reflected in the captured image” depends on an illumination mode (illumination light) at the time of capturing. For example, in images captured under special light such as narrow-band light with a strong short-wavelength component, surface blood vessels are depicted with good contrast, while in images captured under special light with a strong long-wavelength component, deep blood vessels are depicted with good contrast. In addition, the observation and detection (picking up) of a region of interest by a doctor is often performed with normal light (white light) instead of special light.

For example, from JP2014-124333A, there is known for the proper use of illumination light according to the purpose of use of such an image and the target. JP2014-124333A discloses an endoscope device that can switch between a normal light observation mode and a narrow-band light observation mode by an observation mode changeover switch.

### SUMMARY OF THE INVENTION

In observation and diagnosis, medical-use images captured in different illumination modes may be recognized (processed) corresponding to the illumination modes and displayed according to the recognition contents and results. In a case where it is necessary for a user himself/herself to set the image recognition content and display according to the illumination mode, the operation burden is high. However, such a point has not been taken into consideration in the related art such as JP2014-124333A.

The present invention has been made in view of such circumstances, and an object thereof is to provide a medical-use image processing device, an endoscope system, and a medical-use image processing method that can reduce the operation burden on a user.

In order to achieve the above-described object, according to a first aspect of the present invention, there is provided a medical-use image processing device comprising: an image acquisition unit that acquires a medical-use image; a determination unit that determines an illumination mode at the time when the medical-use image is captured; a recognition unit that performs a first recognition for the medical-use image in a case where determination is made that the illumination mode is a first illumination mode, and performs a second recognition for the medical-use image in a case where determination is made that the illumination mode is a second illumination mode; and a display control unit that causes a display device to display a first display according to a result of the first recognition in a case where the determination is made that the illumination mode is the first illumination mode, and causes the display device to display a second display according to a result of the second recognition in a case where the determination is made that the illumination mode is the second illumination mode.

In the first aspect, the determination unit determines the illumination mode, the recognition unit performs the first recognition or the second recognition according to the determination result, and the display control unit causes the display device to display the first display or the second display according to the recognition result. Therefore, it is not necessary for the user himself/herself to set the image recognition content and display according to the illumination mode, and the operation burden on the user can be thus reduced.

In the first aspect, the medical-use image may be captured and acquired at the time of recognition, or an image captured in advance may be acquired. That is, the acquisition, recognition, and display of the image may be performed in parallel, or the image captured and recorded in advance may be subsequently recognized and displayed. Further, the medical-use image acquired by the image acquisition unit may be an image obtained by subjecting the captured image to image processing (emphasis on a specific subject or a specific color component (frequency band), and the like). The medical-use image processing device according to the first aspect can be realized as, for example, a processor of an image diagnosis support system or an endoscope system, or a computer for medical-use image processing, but the present invention is not limited to such an aspect.

The medical-use image processing device according to the first aspect may comprise a repeat control unit that continues processing (determination, recognition, display) of a plurality of medical-use images until the end condition is satisfied. Further, in the first aspect and each of the following aspects, the medical-use image is also referred to as a medical image.

In a second aspect of the medical-use image processing device according to the first aspect, the image acquisition unit acquires the medical-use image in time series, the determination unit makes the determination on a frame constituting the medical-use image acquired in the time series, the recognition unit switches between the first recognition and the second recognition according to a result of the determination being switched between the first illumination mode and the second illumination mode, and the display control unit switches between the first display and the second display according to switching between the first recognition and the second recognition. According to the second aspect, the determination unit switches between the first recognition and the second recognition according to the result of the determination being switched between the first illumination mode and the second illumination mode, and the display control unit switches between the first display and the second display according to switching between the first recognition and the second recognition. Therefore, the user does not need to switch the recognition and display according to the switching of the illumination mode, and the operation burden can be thus reduced by reflecting the user's intention of “which recognition and display are to be performed”. In the second aspect, “acquiring the medical-use image in time series” includes, for example, acquiring a plurality of frames of medical-use images at a predetermined frame rate.

In a third aspect of the medical-use image processing device according to the first or second aspect, the recognition unit detects a region of interest reflected in the medical-use image in the first recognition, and classifies (differentiates) the medical-use image in the second recognition. Since the illumination light generally used differs between the detection and the classification (differentiation), it is preferable to perform different recognition according to the determination result of the illumination mode as in the third aspect. In the third aspect, the classification can be performed on all or a part of the medical-use image regardless of the result of the first recognition (detection). In the third aspect and each of the following aspects, the “region of interest” (ROI) is also referred to as a “region of attention”.

In a fourth aspect of the medical-use image processing device according to the third aspect, the recognition unit performs classification on the region of interest detected in the first recognition, in the second recognition. The fourth aspect defines the target of the second recognition.

In a fifth aspect of the medical-use image processing device according to the third or fourth aspect, the display control unit causes the display device to display information indicating a detection position of the region of interest reflected in the medical-use image in the first display, and causes the display device to display information indicating a classification result of the medical-use image in the second display. As a mode for displaying “information indicating the detection position of the region of interest (first information)”, for example, it is possible to display figures and symbols according to the detection position of the region of interest in a superimposed manner, display the position coordinates numerically, change the color and gradation of the region of interest, and the like. Thus, the user can easily recognize the detection position. Further, as a mode for displaying “information indicating the classification result of the medical-use image (second information)”, for example, characters, numbers, figures, symbols, colors, and the like according to the classification result can be used. Thus, the user can easily recognize the classification result. The first and second information may be superimposed and displayed on the image, or may be displayed separately from the image (displayed in a separate area, displayed on a separate screen, or the like).

In a fifth aspect of the medical-use image processing device according to the third or fourth aspect, the recognition unit includes; a first recognizer that is constructed by learning and performs the first recognition, the first recognizer detecting the region of interest from the medical-use image; and a second recognizer that is constructed by learning and performs the second recognition, the second recognizer classifying the medical-use image. As the first and second recognizers, for example, a trained model constructed by machine learning such as deep learning can be used.

In a sixth aspect of the medical-use image processing device according to the fifth aspect, the first recognizer and the second recognizer have a hierarchical network structure. The sixth aspect defines an example of the configuration of the first and second recognizers, and an example of the “hierarchical network structure” includes a network structure in which an input layer, an interlayer, and an output layer are connected.

In a seventh aspect of the medical-use image processing device according to any one of the first to sixth aspects, the medical-use image processing device further comprises a reception unit that receives an operation of a user, and the determination unit makes the determination based on the received operation. The reception unit can receive, for example, an operation on an operation member for switching the illumination mode.

In an eighth aspect of the medical-use image processing device according to any one of the first to sixth aspects, the determination unit analyzes the acquired medical-use image to make the determination. According to the eighth aspect, even in a case where the information of the user's operation (illumination mode setting, switching, or the like) cannot be acquired, the medical-use image can be analyzed and the determination can be made.

In a ninth aspect of the medical-use image processing device according to the eighth aspect, the determination unit performs the analysis based on a distribution of color components in the medical-use image. The ninth aspect defines an example of a method of analyzing the medical-use image, and focuses on the fact that the distribution of color components in the medical-use image differs depending on the illumination mode (frequency band of illumination light, or the like).

In a tenth aspect of the medical-use image processing device according to the eighth aspect, the determination unit performs the analysis using a convolutional neural network. The convolutional neural network (CNN) is another example of a method of analyzing the medical-use image, and can be constructed by machine learning such as deep learning.

In an eleventh aspect of the medical-use image processing device according to any one of the first to sixth aspects, the determination unit analyzes information displayed on the display device together with the medical-use image to make the determination. The “information displayed on the display device together with the medical-use image” includes, for example, characters indicating the illumination mode, markers such as a frame surrounding the region of interest, numerical values indicating the position coordinates of the region of interest, and characters indicating the classification result of the medical-use image. However, the present invention is not limited thereto. Such an aspect can be used, for example, in a case where the medical-use image processing device cannot directly acquire information on the illumination mode from the image acquisition portion (imaging unit or the like).

In order to achieve the above-described object, according to a twelfth aspect of the present invention, there is provided an endoscope system comprising: the medical-use image processing device according to any one of the first to eleventh aspects; the display device; an endoscope including an insertion part to be inserted into a test object and a hand operation part connected to a proximal end of the insertion part, the insertion part including a distal end rigid portion, a bendable portion connected to a proximal end of the distal end rigid portion, and a flexible portion connected to a proximal end of the bendable portion; a light source device having the first illumination mode and the second illumination mode, the light source device irradiating the test object with first illumination light in the first illumination mode, and irradiating the test object with second illumination light in the second illumination mode; and an imaging unit including an imaging lens that forms an optical image of the test object, and an imaging element on which the optical image is formed by the imaging lens. According to the twelfth aspect, a series of processes from image acquisition to illumination mode determination, image recognition, and display can be performed in the endoscope system. Further, since the endoscope system according to the twelfth aspect comprises the medical-use image processing device according to any one of the first to eleventh aspects, in the series of processes described above, it is not necessary for the user himself/herself to set the image recognition content and display according to the illumination mode, and the operation burden on the user can be thus reduced.

In the twelfth aspect, the light emitted from the light source may be used as it is as the illumination light, or the light generated by applying a filter that transmits a specific wavelength range to the light emitted from the light source may be used as the illumination light. For example, in a case where the narrow-band light is used as the first illumination light and/or the second illumination light, the light emitted from the light source for narrow-band light may be used as the illumination light, or the light generated by applying a filter that transmits a specific wavelength range to the white light may be used as the illumination light. In this case, different narrow-band light beams may be emitted at different timings by sequentially switching the filters applied to the white light.

In a thirteenth aspect of the endoscope system according to the twelfth aspect, the light source device irradiates the test object with normal light as the first illumination light and irradiates the test object with special light as the second illumination light. For example, normal light can be white light including light in the red, blue, and green wavelength ranges, and special light can be narrow-band light corresponding to any wavelength range of red, blue, green, violet, and infrared. However, the present invention is not limited to these examples. According to the thirteenth aspect, for example, in the first illumination mode, detection (first recognition) can be performed on an image captured by white light, and in the second illumination mode, classification (differentiation, second recognition) can be performed on an image captured by special light such as narrow-band light.

In a fourteenth aspect of the endoscope system according to the thirteenth aspect, the light source device comprises: a white light laser light source that emits a white light laser as excitation light; a phosphor that emits white light as the normal light when irradiated with the white light laser; and a narrow-band light laser light source that emits narrow-band light as the special light. The fourteenth aspect defines an example of the configuration of the light source device, and shows an aspect of switching the illumination light by switching the laser light source.

In a fifteenth aspect of the endoscope system according to the thirteenth aspect, the light source device comprises: a white light source that emits white light as the normal light; a white light filter that transmits the white light; a narrow-band light filter that transmits a component of narrow-band light, as the special light, of the white light; and a first filter switching control unit that inserts the white light filter or the narrow-band light filter into an optical path of the white light emitted by the white light source. The fifteenth aspect defines another example of the configuration of the light source device, and shows an aspect of switching the illumination light by inserting a filter into the optical path of the white light.

In a sixteenth aspect of the endoscope system according to the twelfth aspect, the light source device irradiates the test object with first special light as the first illumination light and irradiates the test object with second special light different from the first special light as the second illumination light. The sixteenth aspect defines an aspect in which a plurality of special light beams are used as the illumination light, and for example, a combination of a plurality of blue narrow-band light beams having different wavelengths, blue narrow-band light and green narrow-band light, a plurality of red narrow-band light beams having different wavelengths, and the like can be used. However, the present invention is not limited to these combinations. Narrow-band light corresponding to the violet and/or infrared wavelength range may be used. In the sixteenth aspect, for example, in a case where at least one of the wavelength range or the spectral spectrum of the first special light and the second special light is not the same, it can be determined that it corresponds to “the first special light and the second special light are different”.

In a seventeenth aspect of the endoscope system according to the sixteenth aspect, the light source device comprises: a white light source that emits white light including light in red, blue, and green wavelength ranges; a first narrow-band light filter that transmits a component of first narrow-band light of the white light; a second narrow-band light filter that transmits a component of second narrow-band light of the white light; and a second filter switching control unit that inserts the first narrow-band light filter or the second narrow-band light filter into an optical path of the white light emitted by the white light source. The seventeenth aspect defines still another example of the configuration of the light source device, and shows an aspect of switching the illumination light (narrow-band light) by inserting another filter into the optical path of the white light.

In order to achieve the above-described object, according to an eighteenth aspect of the present invention, there is provided a medical-use image processing method comprising: an image acquisition step of acquiring a medical-use image; a determination step of determining an illumination mode at the time when the medical-use image is captured; a recognition step of performing a first recognition for the medical-use image in a case where determination is made that the illumination mode is a first illumination mode, and performing a second recognition for the medical-use image in a case where determination is made that the illumination mode is a second illumination mode; and a display control step of causing a display device to display a first display according to a result of the first recognition in a case where the determination is made that the illumination mode is the first illumination mode, and causing the display device to display a second display according to a result of the second recognition in a case where the determination is made that the illumination mode is the second illumination mode. According to the eighteenth aspect, the operation burden on the user can be reduced as in the first aspect. The medical-use image processing method according to the eighteenth aspect may comprise a repeat control step of continuing processing (determination, recognition, display) of a plurality of medical-use images until the end condition is satisfied. Further, the medical-use image acquired in the image acquisition step may be an image obtained by subjecting the captured image to image processing (emphasis on a specific subject or a specific color component (frequency band), and the like).

In a nineteenth aspect of the medical-use image processing method according to the eighteenth aspect, the image acquisition step includes acquiring the medical-use image in time series, the determination step includes making the determination on a frame constituting the medical-use image acquired in the time series, the recognition step includes switching between the first recognition and the second recognition according to a result of the determination being switched between the first illumination mode and the second illumination mode, and the display control step includes switching between the first display and the second display in response to switching between the first recognition and the second recognition. According to the nineteenth aspect, as in the second aspect, the user does not need to switch the recognition and display according to the switching of the illumination mode, and the operation burden can be thus reduced by reflecting the user's intention of “which recognition and display are to be performed”.

The medical-use image processing method according to the nineteenth aspect may further include the same configurations as those in the third to eleventh aspects. In addition, a program for causing the medical-use image processing device or the endoscope system to execute the medical-use image processing method of these aspects and a non-transitory recording medium in which a computer-readable code of the program is recorded can also be mentioned as aspects of the present invention.

As described above, according to the medical-use image processing device, the endoscope system, and the medical-use image processing method of the present invention, it is possible to reduce the operation burden on the user.

### BRIEF DESCRIPTION OF THE DRAWINGS

FIG. 1 is an external view illustrating an endoscope system according to a first embodiment.

FIG. 2 is a block diagram showing a configuration of the endoscope system.

FIG. 3 is a diagram showing a configuration of a distal end rigid portion of an endoscope.

FIG. 4 is a diagram showing a functional configuration of an image processing unit.

FIGS. 5A and 5B are diagrams showing a configuration of a determination unit.

FIG. 6 is a diagram showing a configuration of a recognition unit.

FIGS. 7A and 7B are diagrams showing a configuration example of a convolutional neural network.

FIG. 8 is a flowchart showing a procedure of a medical-use image processing method according to the first embodiment.

FIGS. 9A to 9C are diagrams showing an example of a first display.

FIG. 10 is a diagram showing another example of the first display.

FIGS. 11A to 11C are diagrams showing an example of a second display.

FIG. 12 is a diagram showing another example of the second display.

FIG. 13 is a flowchart showing another procedure of the medical-use image processing method according to the first embodiment.

FIG. 14 is a flowchart showing still another procedure of the medical-use image processing method according to the first embodiment.

FIG. 15 is a diagram showing a configuration example of a light source.

FIG. 16 is a diagram showing still another configuration example of the light source.

FIGS. 17A and 17B are diagrams showing an example of a rotation filter.

FIGS. 18A and 18B are diagrams showing another example of the rotation filter.

### DESCRIPTION OF THE PREFERRED EMBODIMENTS

Hereinafter, a medical-use image processing device, an endoscope system, and a medical-use image processing method according to an embodiment of the present invention will be described in detail with reference to the accompanying drawings.

### First Embodiment

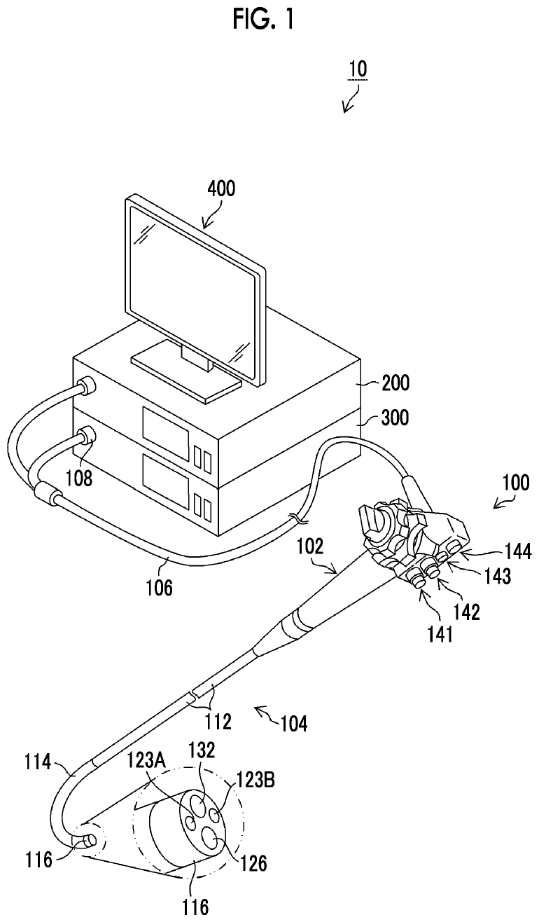

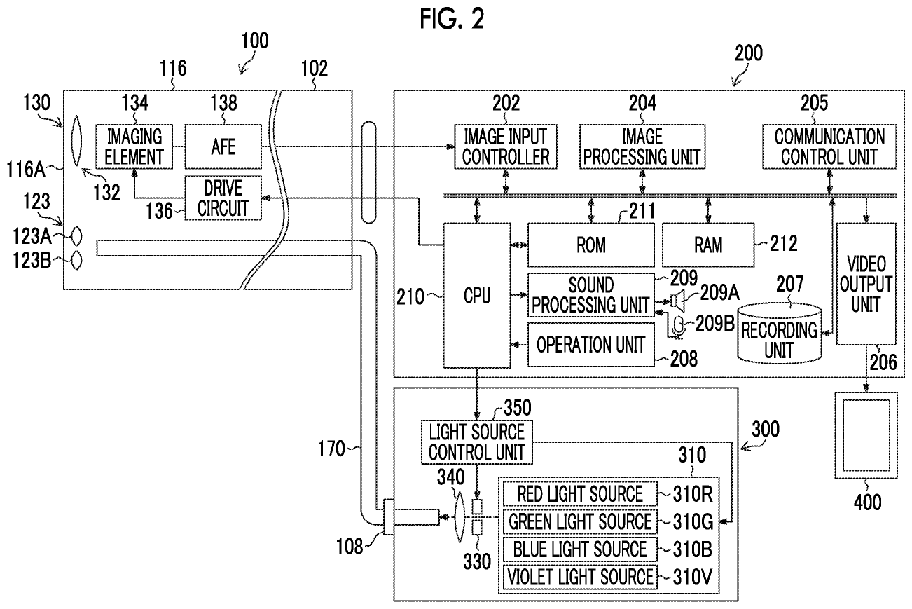

FIG. 1 is an external view showing an endoscope system 10 (medical-use image processing device, medical image processing device, diagnosis support device, endoscope system) according to a first embodiment, and FIG. 2 is a block diagram showing a configuration of the main part of the endoscope system 10. As shown in FIGS. 1 and 2, the endoscope system 10 is configured to include an endoscope main body 100 (endoscope), a processor 200 (processor, image processing device, medical image processing device), a light source device 300 (light source device), and a monitor 400 (display device).

<Configuration of Endoscope Main Body>

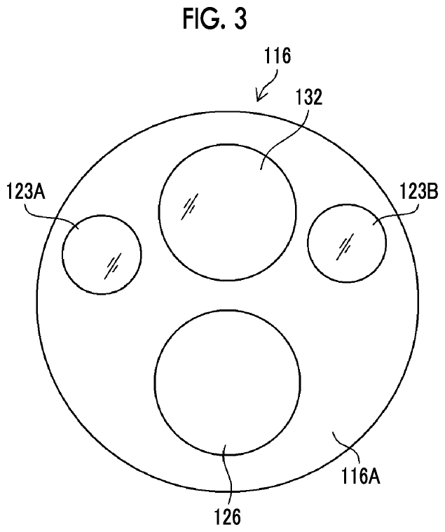

The endoscope main body 100 comprises a hand operation part 102 (hand operation part) and an insertion part 104 (insertion part) connected to the hand operation part 102. An operator (user) grasps and operates the hand operation part 102, and inserts the insertion part 104 into the body of a test object (living body) to observe the test object. In addition, the hand operation part 102 is provided with an air-supply and water-supply button 141, a suction button 142, a function button 143 to which various functions are assigned, and an imaging button 144 that receives imaging start and end instruction operations (still image, video image). A function for setting or switching illumination modes may be assigned to the function button 143. The insertion part 104 includes a flexible portion 112 (flexible portion), a bendable portion 114 (a bendable portion), and a distal end rigid portion 116 (distal end rigid portion) in this order from the hand operation part 102 side. That is, the bendable portion 114 is connected to a proximal end of the distal end rigid portion 116, and the flexible portion 112 is connected to a proximal end of the bendable portion 114. The hand operation part 102 is connected to the proximal end of the insertion part 104. The user can bend the bendable portion 114 by operating the hand operation part 102 to change the direction of the distal end rigid portion 116 vertically and horizontally. The distal end rigid portion 116 is provided with an imaging optical system 130 (imaging unit), an illumination part 123, a forceps port 126, and the like (refer to FIGS. 1 to 3).

During observation and treatment, white light and/or narrow-band light as special light (one or more of red narrow-band light, green narrow-band light, blue narrow-band light, and violet narrow-band light) can be emitted from illuminating lenses 123A and 123B of the illumination part 123 by operating an operation unit 208 (refer to FIG. 2). In addition, by the operation of the air-supply and water-supply button 141, cleaning water is discharged from a water-supply nozzle (not shown), and an imaging lens 132 (imaging lens, imaging unit) and the illuminating lenses 123A and 123B of the imaging optical system 130 can be cleaned. A pipe line (not shown) is communicated with the forceps port 126 opened at the distal end rigid portion 116, and a treatment tool (not shown) for tumor excision or the like is inserted into the pipe line and is appropriately moved forward and backward to perform a necessary treatment on the test object.

As shown in FIGS. 1 to 3, the imaging lens 132 (imaging unit) is arranged on a distal end side end surface 116A of the distal end rigid portion 116. A complementary metal-oxide semiconductor (CMOS) type imaging element 134 (imaging element, imaging unit), a drive circuit 136, and an analog front end (AFE) 138 are arranged at the back of the imaging lens 132, and an image signal is output by these elements. The imaging element 134 is a color image pickup element and comprises a plurality of pixels composed of a plurality of light-receiving elements disposed in a matrix (two-dimensionally arrayed) in a specific pattern array (Bayer array, X-Trans (registered trademark) array, honeycomb array, or the like). Each pixel of the imaging element 134 includes a microlens, a red (R), green (G), or blue (B) color filter, and a photoelectric conversion part (photodiode or the like). The imaging optical system 130 can generate a color image from pixel signals of three colors of red, green, and blue, or can generate an image from pixel signals of any one or two colors of red, green, and blue. In the first embodiment, a case where the imaging element 134 is a CMOS type imaging element will be described, but the imaging element 134 may be a charge coupled device (CCD) type imaging element. Each pixel of the imaging element 134 may further comprise a violet color filter corresponding to a violet light source and/or an infrared filter corresponding to an infrared light source. In this case, the image can be generated in consideration of the violet and/or infrared pixel signals.

An optical image of a test object (tumor part, lesion part) is formed on a light-receiving surface (imaging surface) of the imaging element 134 by the imaging lens 132, converted into an electric signal, output to the processor 200 via a signal cable (not shown), and converted into a video signal. As a result, an observation image is displayed on the monitor 400 connected to the processor 200.

In addition, the illuminating lenses 123A and 123B of the illumination part 123 are provided adjacent to the imaging lens 132 on the distal end side end surface 116A of the distal end rigid portion 116. At the back of the illuminating lenses 123A and 123B, an emission end of a light guide 170 described later is arranged, the light guide 170 is inserted into the insertion part 104, the hand operation part 102, and a universal cable 106, and an incident end of the light guide 170 is disposed in a light guide connector 108.

<Configuration of Light Source Device>

As shown in FIG. 2, the light source device 300 is configured to include a light source 310 for illumination, a stop 330, a condensing lens 340, a light source control unit 350, and the like, and allows illumination light (observation light) to enter the light guide 170. The light source 310 comprises a red light source 310R, a green light source 310G, a blue light source 310B, and violet light source 310V that respectively emit red, green, blue, and violet narrow-band light beams, and can emit with red, green, blue, violet narrow-band light beams. The illuminance of the illumination light by the light source 310 is controlled by the light source control unit 350, and the illuminance of the illumination light can be lowered and the illumination can be stopped as necessary.

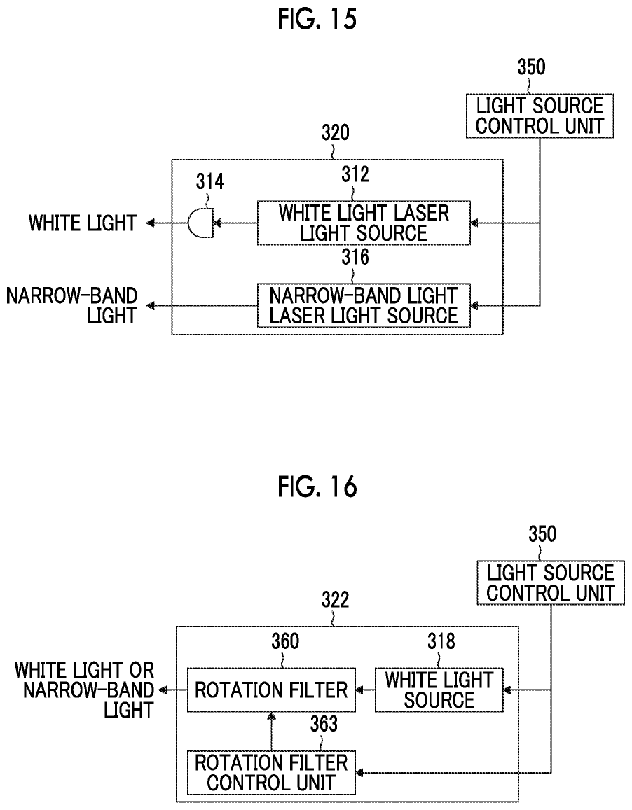

The light source 310 can emit red, green, blue, violet narrow-band light beams in any combination. For example, white light (normal light) can be emitted as illumination light (observation light) by simultaneously emitting red, green, blue, violet narrow-band light beams, or narrow-band light as special light can be emitted by emitting one or two of them. The light source 310 may further comprise an infrared light source that emits infrared light (an example of narrow-band light). In addition, white light or narrow-band light may be emitted as illumination light by a light source that emits white light and a filter that transmits the white light and each narrow-band light (refer to, for example, FIGS. 15 to 18).

<Wavelength Range of Light Source>

The light source 310 may be a light source that generates light in a white range or light in a plurality of wavelength ranges as light in a white range, and alternatively may be a light source that generates light in a specific wavelength range narrower than a white wavelength range. The specific wavelength range may be a blue range or a green range in a visible range, or a red range in a visible range. In a case where the specific wavelength range is the blue range or the green range in the visible range, the specific wavelength range includes a wavelength range of 390 nm to 450 nm, or 530 nm to 550 nm, and may have a peak wavelength in a wavelength range of 390 nm to 450 nm, or 530 nm to 550 nm. In addition, in a case where the specific wavelength range is the red range in the visible range, the specific wavelength range includes a wavelength range of 585 nm to 615 nm, or 610 nm to 730 nm, and light in the specific wavelength range may have a peak wavelength in a wavelength range of 585 nm to 615 nm, or 610 nm to 730 nm.

The light of the specific wavelength range described above includes a wavelength range in which light absorption coefficients of oxygenated hemoglobin and reduced hemoglobin are different, and may have a peak wavelength in the wavelength range in which the light absorption coefficients of oxygenated hemoglobin and reduced hemoglobin are different. In this case, the specific wavelength range includes a wavelength range of 400±10 nm, 440±10 nm, 470±10 nm, or 600 nm to 750 nm, and may have a peak wavelength in a wavelength range of 400±10 nm, 440±10 nm, 470±10 nm, or 600 nm to 750 nm.

In addition, the light generated by the light source 310 includes a wavelength range of 790 nm to 820 nm or 905 nm to 970 nm, and may have a peak wavelength in a wavelength range of 790 nm to 820 nm or 905 nm to 970 nm.

The light source 310 may comprise a light source that emits excitation light having a peak of 390 nm to 470 nm. In this case, a medical-use image (in-vivo image) having information on fluorescence emitted by a fluorescent material in the test object (living body) can be acquired. In a case of acquiring a fluorescence image, a coloring agent for a fluorescence method (such as fluorescein and acridine orange) may be used.

A light source type (a laser light source, a xenon light source, a light-emitting diode (LED) light source, and the like), a wavelength, presence or absence of a filter, and the like of the light source 310 are preferably configured according to a type of the subject, a purpose of observation, and the like. In addition, at the time of observation, the wavelength of the illumination light is preferably combined and/or switched according to a type of the subject, a purpose of observation, and the like. In a case where the wavelength is switched, the wavelength of the light to be emitted may be switched by, for example, rotating a disk-shaped filter (a rotary color filter) that is disposed in front of the light source and provided with a filter that transmits or blocks light having a specific wavelength (refer to FIGS. 15 to 18).

In addition, the imaging element used in carrying out the present invention is not limited to a color image pickup element in which a color filter is arranged for each pixel as in the imaging element 134, and may be a monochrome imaging element. In a case of using the monochrome imaging element, it is possible to capture an image in a field-sequential (color-sequential) manner by sequentially switching the wavelength of illumination light (observation light). For example, the wavelength of the emitted illumination light may be sequentially switched among (violet, blue, green, and red), or the wavelength of the illumination light emitted by the rotary color filter (red, green, blue, violet, or the like) may be switched by emitting broadband light (white light) (refer to a configuration example of the light source described later; FIGS. 16 to 18). In addition, the wavelength of the illumination light emitted by the rotary color filter (green, blue, or the like) may be switched by emitting one or a plurality of narrow-band light beams (green, blue, or the like). The narrow-band light may be infrared light (first narrow-band light, second narrow-band light) having two or more different wavelengths. In the case of capturing an image in a field-sequential (color-sequential) manner in this way, images may be acquired and combined by changing the intensity of illumination light between colors, or images of each piece of color light acquired with the intensity of the illumination light constant between the colors may be weighted and combined.

By connecting the light guide connector 108 (refer to FIG. 1) to the light source device 300, illumination light emitted from the light source device 300 is transmitted to the illuminating lenses 123A and 123B via the light guide 170, and is emitted from the illuminating lenses 123A and 123B to the observation region.

<Configuration of Processor>

A configuration of the processor 200 will be described with reference to FIG. 2. The processor 200 inputs an image signal output from the endoscope main body 100 via an image input controller 202, performs necessary image processing in an image processing unit 204 (medical-use image processing device), and outputs the image signal via a video output unit 206. As a result, an observation image (in-vivo image) is displayed on the monitor 400 (display device). These processes are performed under control of a central processing unit (CPU) 210. That is, the CPU 210 has functions as an image acquisition unit, a determination unit, a recognition unit, a display control unit, a reception unit, and a repeat control unit. A communication control unit 205 controls communication with an in-hospital system (hospital information system (HIS)), an in-hospital local area network (LAN), and the like, which are not shown. A recording unit 207 records an image (medical-use image, captured image) of the subject, information indicating the result of detection and/or classification of the region of interest, and the like. Under the control of the CPU 210 and the image processing unit 204, a sound processing unit 209 outputs a message (sound) or the like according to the result of detection and/or classification of a region of interest from a speaker 209A. Further, the sound processing unit 209 (medical-use image processing device, reception unit) can collect the user's voice by a microphone 209B and recognize what kind of operation (illumination mode setting, switching operation, or the like) has been performed. That is, the sound processing unit 209 and the microphone 209B function as a reception unit for receiving operations of the user.

A read only memory (ROM) 211 is a nonvolatile storage element (non-transitory recording medium), and stores a computer-readable code of a program for causing the CPU 210 and/or the image processing unit 204 (medical-use image processing device, computer) to execute the medical-use image processing method according to the embodiment of the present invention. A random access memory (RAM) 212 is a storage element for temporary storage during various types of processing, and can also be used as a buffer during image acquisition.

<Function of Image Processing Unit>

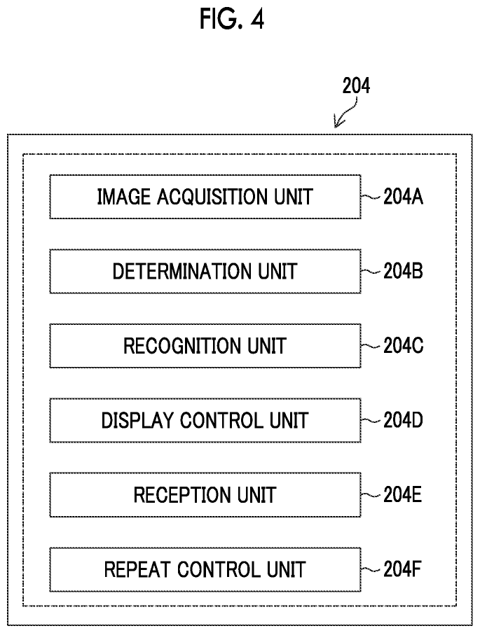

FIG. 4 is a diagram showing a functional configuration of the image processing unit 204 (medical-use image processing device, medical image acquisition unit, medical image analysis processing unit, medical image analysis result acquisition unit). The image processing unit 204 includes an image acquisition unit 204A (image acquisition unit), a determination unit 204B (determination unit), a recognition unit 204C (recognition unit), a display control unit 204D (display control unit), a reception unit 204E (reception unit), and a repeat control unit 204F (repeat control unit). The determination unit 204B and the recognition unit 204C also operate as a medical image analysis processing unit.

The image processing unit 204 may comprise a special light image acquisition unit that acquires a special light image having information on a specific wavelength range on the basis of a normal light image obtained by emitting light in a white range or light in a plurality of wavelength ranges as light in a white range. In this case, the signal of the specific wavelength range can be obtained by calculation based on color information of RGB (R: red, G: green, B: blue) or CMY (C: cyan, M: magenta, Y: yellow) included in the normal light image.

The image processing unit 204 may comprise a feature quantity image generation unit that generates a feature quantity image by calculation based on at least one of a normal light image obtained by emitting light in a white range or light in a plurality of wavelength ranges as light in a white range or a special light image obtained by emitting light in a specific wavelength range, and may acquire and display the feature quantity image as a medical-use image (medical image). The display control unit 204D may have the function of the feature quantity image generation unit. Further, the image processing unit 204 may comprise a signal processing unit that emphasizes colors in a specific wavelength range by signal processing (for example, performing color expansion and/or reduction in the color space so that reddish colors are redder and whitish colors are whiter, and emphasizing subtle color differences in mucous membranes).

<Configuration of Determination Unit>

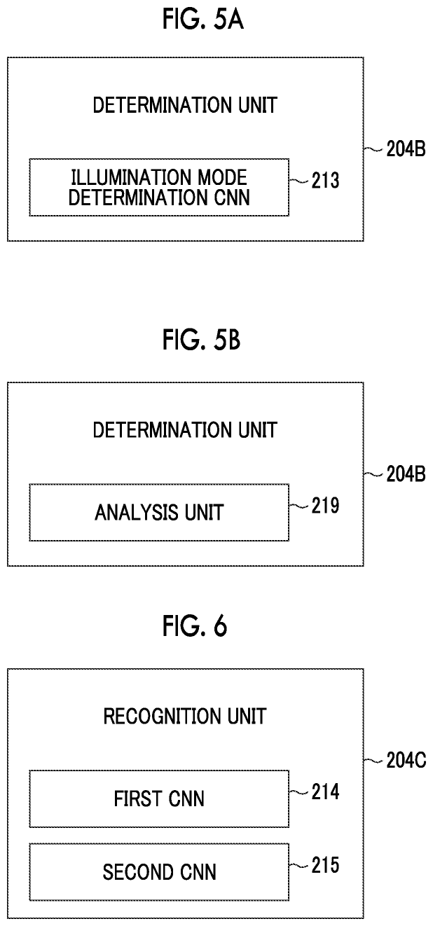

As shown in FIG. 5A, the determination unit 204B includes an illumination mode determination CNN 213 (CNN: convolutional neural network). The illumination mode determination CNN 213 has a hierarchical network structure, and analyzes the acquired medical-use image to determine the illumination mode (details will be described later). In addition to or instead of the illumination mode determination CNN 213, an analysis unit 219 may be provided as shown in FIG. 5B, and the determination may be made based on the analysis by the analysis unit 219 (user operations received by the reception unit 204E, distribution of color components in the acquired medical-use image, analysis based on information displayed on the monitor 400 together with the medical-use image, and the like).

<Configuration of Recognition Unit>

As shown in FIG. 6, the recognition unit 204C includes a first CNN 214 (first recognizer) and a second CNN 215 (second recognizer). The first CNN 214 and the second CNN 215 are convolutional neural networks similar to the above-mentioned illumination mode determination CNN 213, and have a hierarchical network structure. The first CNN 214 is a first recognizer that is constructed by learning and performs the first recognition, and detects the region of interest from the medical-use image. Further, the second CNN 215 is a second recognizer that is constructed by learning and performs the second recognition, and classifies (differentiates) the medical-use image. The recognition unit 204C can determine which CNN to use according to the determination result of the illumination mode.

<Layer Configuration of CNN>

The layer configuration of the above-mentioned CNN (illumination mode determination CNN 213, first CNN 214, second CNN 215) will be described. Hereinafter, the first CNN 214 will be mainly described, but the same configuration can be adopted for the second CNN 215 and the illumination mode determination CNN 213.

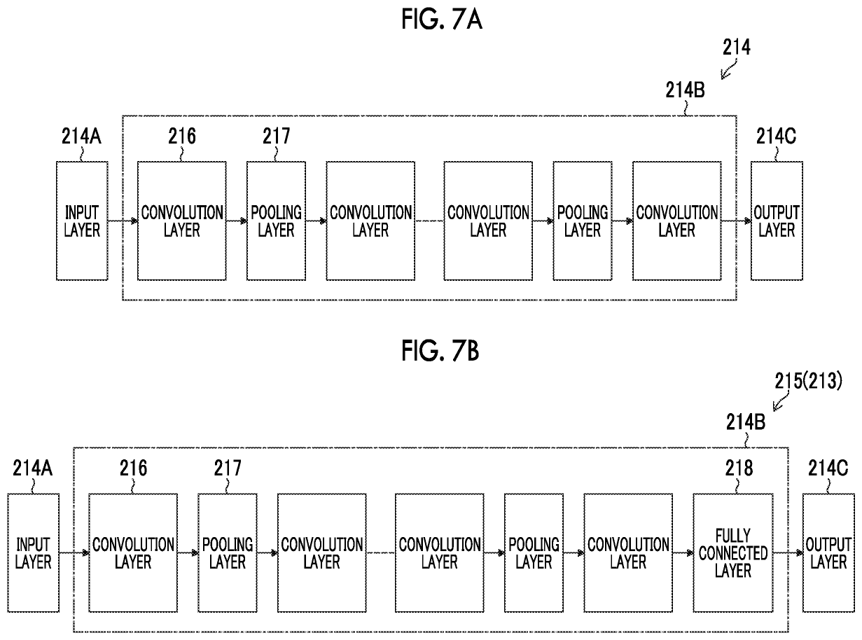

FIGS. 7A and 7B are diagrams showing an example of the layer configuration of CNN. In the example shown in FIG. 7A, the first CNN 214 includes an input layer 214A, an interlayer 214B, and an output layer 214C. The input layer 214A inputs an image (for example, a normal light image) captured in the first illumination mode and outputs a feature quantity. The interlayer 214B includes a convolution layer 216 and a pooling layer 217, and the feature quantity output by the input layer 214A is input to calculate other feature quantities. These layers have a structure in which a plurality of “nodes” are connected by “edges” and hold a plurality of weight parameters. The value of the weight parameter changes as the learning progresses. The layer configuration of the first CNN 214 is not limited to the case where the convolution layer 216 and the pooling layer 217 are repeated one by one, and a plurality of layers (for example, convolution layer 216) may be continuously included.

<Processing in Interlayer>

The interlayer 214B calculates the feature quantity by a convolution operation and a pooling process. The convolution operation performed in the convolution layer 216 is a process of acquiring a feature map by a convolution operation using a filter, and plays a role of feature extraction such as edge extraction from an image. By the convolution operation using this filter, one channel (one sheet) of “feature map” is generated for one filter. The size of the “feature map” is downscaled by convolution and becomes smaller as each layer is convolved. The pooling process performed in the pooling layer 217 is a process of reducing (or enlarging) the feature map output by the convolution operation to make a new feature map, and plays a role of imparting robustness so that the extracted features are not affected by translation or the like. The interlayer 214B can be composed of one or a plurality of layers that perform these processes.

Among the layers of the interlayer 214B, lower-order feature extraction (edge extraction or the like) is performed in the convolution layer closer to the input side, and higher-order feature extraction (extraction of features related to the shape, structure, and the like of the object) is performed as it approaches the output side. In a case where segmentation is performed, the convolution layer in the latter half is upscaled, and in the last convolution layer, a “feature map” of the same size as the input image set is obtained. On the other hand, in the case of performing object detection, upscaling is not essential because position information is only required to be output.

The interlayer 214B may include a layer for batch normalization in addition to the convolution laver 216 and the pooling layer 217. The batch normalization process is a process of normalizing the distribution of data in units of mini-batch when learning, and plays a role of advancing learning quickly, reducing dependence on initial values, suppressing overtraining, and the like.

<Processing in Output Layer>

The output layer 214C is a layer that detects the position of the region of interest reflected in the input medical-use image (normal light image, special light image) based on the feature quantity output from the interlayer 214B and outputs the result. Since the first CNN 214 performs segmentation, the output layer 214C grasps the position of the region of interest reflected in the image at the pixel level by the “feature map” obtained from the interlayer 214B. That is, it is possible to detect whether or not each pixel of the endoscopic image belongs to the region of interest and output the detection result. In the case of detecting an object, it is not necessary to make determination at the pixel level, and the output laver 214C outputs the position information of the object.

In the second CNN 215, the output layer 214C executes the classification (differentiation; second recognition) of the medical-use image and outputs the classification result. For example, the output layer 214C classifies endoscopic images into three categories of “neoplastic”, “non-neoplastic”, and “other”, and may output a differentiation result as three scores (the sum of the three scores is 100%) corresponding to “neoplastic”, “non-neoplastic” and “other” or may output a classification result in a case where it can be clearly classified from the three scores. Similarly, the illumination mode determination CNN 213 determines the illumination mode of the medical-use image and outputs the determination result (for example, “normal light (white light) mode”, “first special light (narrow-band light) mode”, “second special light (narrow-band light) mode”). In a case where the classification result is output as in the second CNN 215 and the illumination mode determination CNN 213, it is preferable that the output layer 214C includes a fully connected layer 218 as the last one or more layers (refer to FIG. 7B). For the other layers, the same configuration as the first CNN 214 described above can be used.

The first CNN 214 having the above-described configuration can be constructed by learning using images and information regarding the position of the region of interest in the images (for example, machine learning such as deep learning). Similarly, the second CNN 215 can be constructed by learning using images and information regarding categories of the images. Further, the illumination mode determination CNN 213 can be constructed by learning using images and information regarding the illumination mode of the images.

<Realization of Function of Image Processing Unit by Processor, Etc.>

The function of the image processing unit 204 described above can be realized by using various processors. The various processors include, for example, a central processing unit (CPU) that is a general-purpose processor that executes software (program) to realize various functions. In addition, the above-described various processors include a programmable logic device (PLD) which is a processor whose circuit configuration can be changed after manufacturing, such as a graphics processing unit (GPU) and a field programmable gate array (FPGA) which are processors specialized for image processing. Further, the above-described various processors also include a dedicated electric circuit which is a processor having a circuit configuration designed exclusively for executing specific processing such as an application specific integrated circuit (ASIC).

The function of each unit may be realized by one processor, or may be realized by a plurality of processors of the same type or different types (for example, a plurality of FPGAs, a combination of a CPU and an FPGA, or a combination of a CPU and a GPU). In addition, a plurality of functions may be realized by one processor. As a first example in which the plurality of functions are configured by one processor, there is an aspect in which one processor is configured by a combination of one or more CPUs and software, and the processor is realized as the plurality of functions, as represented by a computer such as an image processing device main body or a server. As a second example, there is an aspect in which a processor for realizing the functions of the entire system by one integrated circuit (IC) chip as represented by a system on chip (SoC) or the like is used. In this way, various functions are configured by using one or more of the above-described various processors as a hardware structure. Furthermore, the hardware structure of these various processors is, more specifically, an electric circuit (circuitry) in which circuit elements such as semiconductor elements are combined. These electric circuits may be electric circuits that realize the above-mentioned functions by using logical sum, logical product, logical denial, exclusive OR, and logical operations combining these.

In a case where the above-described processor or electric circuit executes software (program), a processor-(computer-) readable code of the software to be executed is stored on a non-transitory recording medium such as a read only memory (ROM), and the processor refers to the software. Software stored on a non-transitory recording medium includes programs for executing medical-use image acquisition, illumination mode determination, first and second recognitions, and display control. The code may be recorded on a non-transitory recording medium such as various types of magneto-optical recording device or a semiconductor memory instead of the ROM. In the processing using the software, for example, a random access memory (RAM) is used as a temporary storage area, and data stored in, for example, an electronically erasable and programmable read only memory (EEPROM) (not shown) can be referred to.

Details of the processing by these functions of the image processing unit 204 will be described later. The processing by these functions is performed under the control of the CPU 210.

<Configuration of Operation Unit>

The processor 200 comprises the operation unit 208 (reception unit). The operation unit 208 comprises an illumination mode setting switch, a foot switch, and the like (not shown), and can set the illumination mode (whether to use normal light (white light), special light such as narrow-band light, or narrow-band light of which wavelength in the case of narrow-band light). Further, the operation unit 208 includes a keyboard and a mouse (not shown), and the user can perform, via these devices, setting operations of imaging conditions and display conditions, setting and switching operations of the illumination mode, and imaging instructions (acquisition instructions) of a video image or a still image (the imaging instructions of the video image and the still image may be given by the imaging button 144). These setting operations may be performed via the foot switch or the like described above, or may be performed by sound (which can be processed by the microphone 209B and the sound processing unit 209), line of sight, gesture, or the like. That is, the operation unit 208 functions as a reception unit for receiving operations of the user.

<Configuration of Recording Unit>

The recording unit 207 (recording device) is configured to include various magneto-optical magnetic recording media, a non-transitory recording medium such as a semiconductor memory, and a control unit of these recording media, and can record the endoscopic image (medical-use image, medical image), the setting information and the determination result of the illumination mode, the detection result of the region of interest (result of first recognition), the classification result of the medical-use image (differentiation result; result of second recognition), and the like in association with each other. These images and information are displayed on the monitor 400 by an operation via the operation unit 208 and under control of the CPU 210 and/or the image processing unit 204.

<Configuration of Display Device>

The monitor 400 (display device) displays an endoscopic image, a determination result of an illumination mode, a detection result of a region of interest, a classification result of a medical-use image, and the like by an operation via the operation unit 208 and under control of the CPU 210 and/or the image processing unit 204. In addition, the monitor 400 has a touch panel (not shown) for performing an imaging condition setting operation and/or a display condition setting operation.

<Medical-Use Image Processing Method>

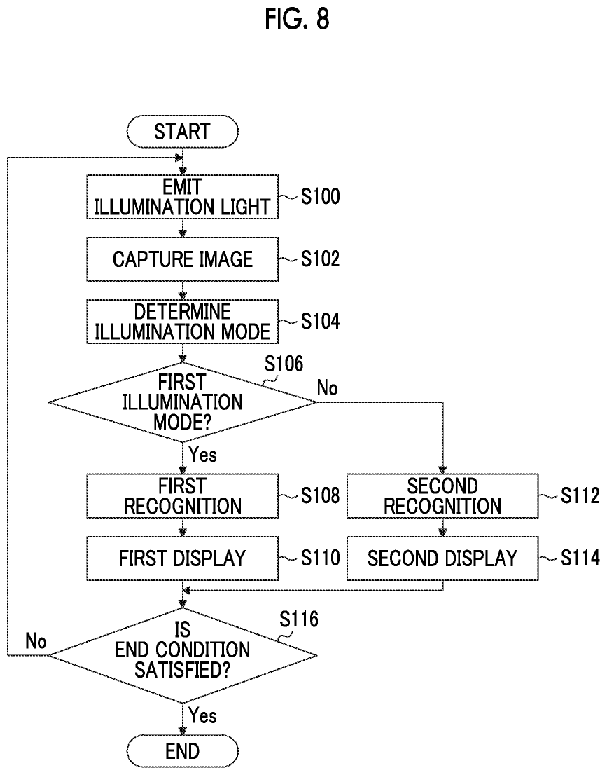

The medical-use image processing method using the endoscope system 10 having the above-described configuration will be described. FIG. 8 is a flowchart showing a procedure of a medical-use image processing method according to the first embodiment.

<Acquisition of Medical-Use Images>

In Step S100, the light source device 300 emits the illumination light according to the setting (setting and switching of the illumination mode) via the operation unit 208 or the like. Here, a case of emitting white light (normal light) as the first illumination light or blue narrow-band light (special light, narrow-band light) as the second normal light will be described, but the combination of illumination light beams is not limited to this example. An image (medical-use image) of a test object is captured by the imaging optical system 130 under the first illumination light or the second illumination light, and the captured image is acquired by the image acquisition unit 204A (image acquisition step). The image acquisition unit 204A can acquire medical-use images in time series at a predetermined frame rate.

<Determination of Illumination Mode>

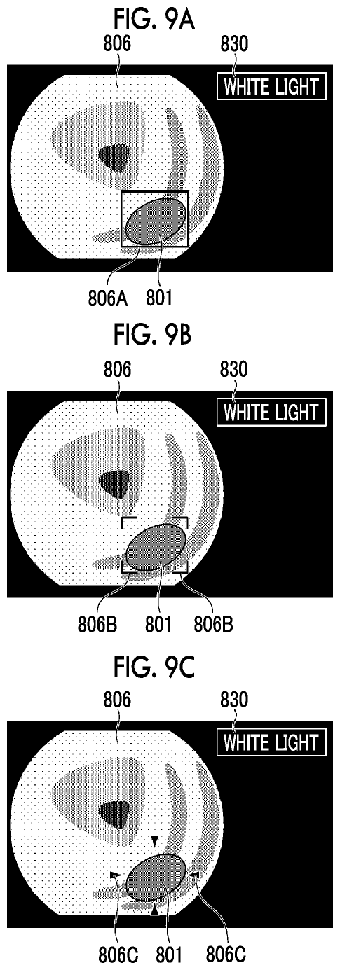

In the determination unit 204B, the illumination mode determination CNN 213 analyzes the medical-use image (classification described above) and determines the illumination mode (Step S104: determination step). Further, the determination unit 204B may analyze the medical-use image by the analysis unit 219 described above to determine the illumination mode. In a case where the analysis unit 219 performs the analysis, the reception unit 204E (reception unit) receives the operation of the user (setting and switching of the illumination mode), and the determination can be made based on the received operation. The user can operate the microphone 209B, the sound processing unit 209, the function button 143 provided on the hand operation part 102 (as described above, the function of setting or switching the illumination mode can be assigned), a keyboard and mouse (not shown) of the operation unit 208, an illumination mode setting switch, a foot switch, and the like (not shown). In addition, the analysis unit 219 may perform analysis based on the distribution of color components in the acquired medical-use image to determine the illumination mode. Further, the analysis unit 219 may analyze the information (refer to FIGS. 9A to 12) displayed on the monitor 400 (display device) together with the medical-use image to determine the illumination mode.

<Recognition and Display Control>

In a case where it is determined as a result of Step S104 that “the illumination mode is the first illumination mode” (YES in Step S106), a first recognition and a first display are performed in steps S108 and S110, respectively (recognition step, display control step). On the other hand, in a case where it is determined as a result of Step S104 that “the illumination mode is the second illumination mode” (NO in Step S106), a second recognition and a second display are performed in steps S112 and S114, respectively (recognition step, display control step).

<First Recognition and First Display>

The recognition unit 204C detects the region of interest reflected in the medical-use image by performing the segmentation described above by the first CNN 214 (first recognizer) (Step S108; recognition step, first recognition). Examples of the region of interest (region of attention) detected in Step S108 include polyps, cancers, diverticula of the large intestine, inflammations, treatment scars (endoscopic mucosal resection (EMR) scar, endoscopic submucosal dissection (ESD) scar, clip portions, and the like), bleeding points, perforations, angiodysplasia, and the like.

According to the result of the first recognition, the display control unit 204D causes the monitor 400 (display device) to display the first display (Step S10: display control step). FIGS. 9A to 9C are diagrams showing an example of the first display, as shown in FIGS. 9A to 9C with respect to a region of interest 801 reflected in a medical-use image 806, respectively, a frame 806A, a marker 806B, and a marker 806C (examples of information indicating the detection position of the region of interest) surrounding the region of interest 801 are displayed. Further, the display control unit 204D displays the type of illumination light, the illumination mode, and the like in a region 830 based on the result of the above-described determination. Although it is displayed as “white light” in FIGS. 9A to 9C, it may be “first illumination mode”, “white light (normal light) mode”, or the like. Further, the recognition content (“first recognition”, “detection of the region of interest”, or the like) may be displayed. The type of illumination light, the illumination mode, the recognition content, and the like are examples of information displayed on the display device together with the medical-use image. The recognition unit 204C may provide information indicating the detection result of the region of interest by sound via the sound processing unit 209 and the speaker 209A.

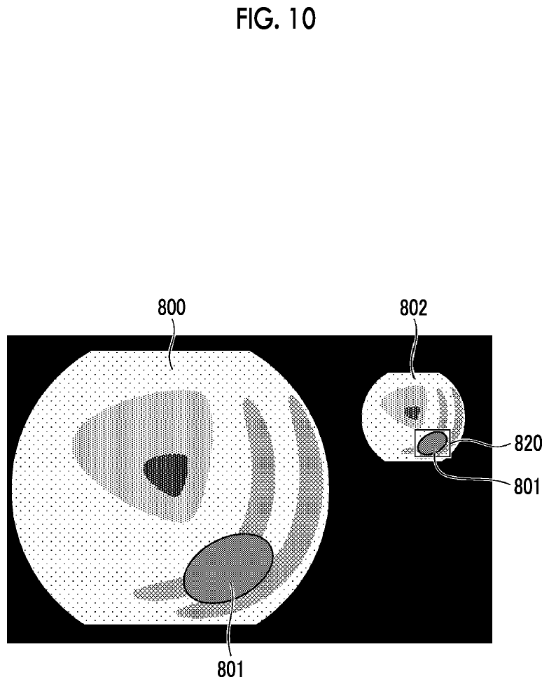

FIG. 10 is a diagram showing another example of the first display. FIG. 10 shows a mode in which a medical-use image 800 constituting each frame of the medical-use images acquired in time series is continuously displayed, and a medical-use image 802 in which a region of interest 801 is detected and a frame 820 is displayed is frozen and displayed (a target frame is continuously displayed separately from the medical-use images acquired in time series). In a case where another region of interest is detected, a freeze display may be added (multiple displayed). Further, in a case where a certain time has passed after the display or in a case where there is no empty portion in the display area of the monitor 400, the freeze display may be deleted. Even in the case of performing such a freeze display, the type of illumination light, the illumination mode, the recognition content, and the like may be displayed as in FIGS. 9A to 9C.

The recognition unit 204C may detect the region of interest by a method other than CNN. For example, the region of interest can be detected based on the feature quantity of the pixels of the acquired medical-use image. In this case, the recognition unit 204C divides a detection target image into, for example, a plurality of rectangular regions, sets each of the divided rectangular regions as a local region, calculates the feature quantity (for example, hue) of the pixels in the local region for each local region of the detection target image, and determines a local region having a specific hue from each local region as the region of interest.

<Second Recognition and Second Display>

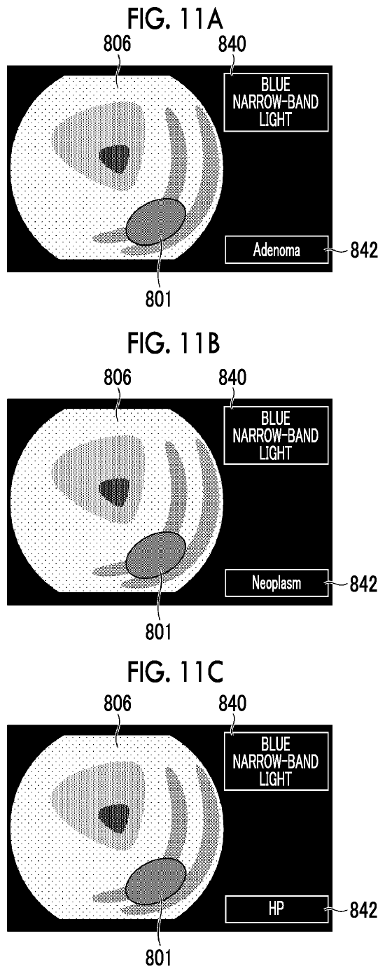

The recognition unit 204C classifies (differentiates) medical-use images by the second CNN 215 (second recognizer) (Step S112: recognition step, second recognition). The classification can be performed on all or a part of the medical-use image regardless of the result of the first recognition (detection) described above, but the classification may be performed on the region of interest detected by the first recognition. The recognition unit 204C may determine the range of the target to be classified based on the users instruction operation via the operation unit 208, or without the user's instruction operation. Examples of classification include the type of lesion (hyperplastic polyp, adenoma, intramucosal cancer, invasive cancer, or the like), extent of lesion, size of lesion, macroscopic shape of lesion, diagnosis of the stage of cancer, current location in the lumen (pharynx, esophagus, stomach, duodenum, or the like in the upper part, and cecum, ascending colon, transverse colon, descending colon, sigmoid colon, rectum, or the like in the lower part), and the like. According to the result of the second recognition, the display control unit 204D causes the monitor 400 (display device) to display the second display (Step S114: display control step). FIGS. 1I A to 11C are diagrams showing an example of the second display, and the classification result of the medical-use image 806 is displayed in a region 842. FIGS. 11A to 11C show examples in the case where the classification results are adenoma, neoplasm (tumor), and Helicobacter pylori (HP), respectively. The display control unit 204D may display information indicating the reliability of the classification result (which can be calculated by the second CNN 215) by numerical values, figures (for example, bar display), symbols, colors, and the like. Further, the recognition unit 204C may provide information indicating the classification result by sound via the sound processing unit 209 and the speaker 209A.

Further, the display control unit 204D displays the type of illumination light, the illumination mode, and the like in a region 840 based on the result of the above-described determination, similarly to the region 830 of FIGS. 9A to 9C. Although it is displayed as “blue narrow-band light” in FIGS. 11A to 11C, it may be “second illumination mode”, “special light (narrow-band light) mode”, or the like. Further, the recognition content (“second recognition”, “classification of the medical-use image”, or the like) may be displayed. The information displayed in the regions 840 and 842 (the type of illumination light, the illumination mode, the recognition content, the classification result, and the like) is an example of information displayed on the display device together with the medical-use image.

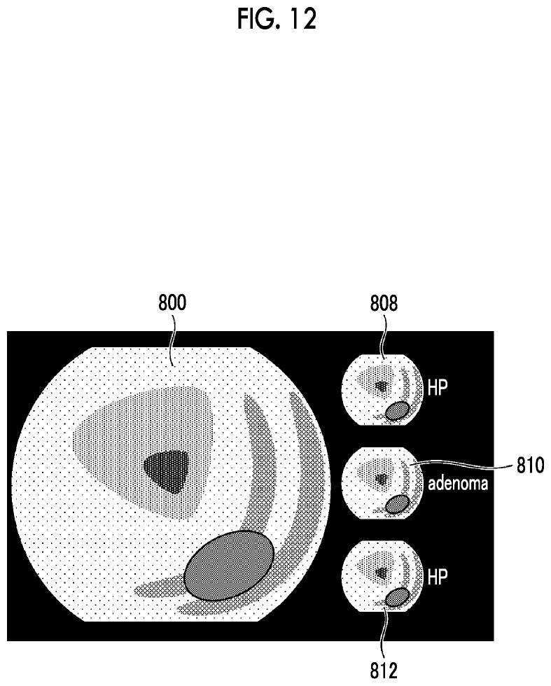

In the second display as well, the freeze display may be performed as in the case of the first display. FIG. 12 is an example of the freeze display in the second display, and shows a state in which the medical-use image 800 constituting each frame of the medical-use images acquired in time series is continuously displayed, and medical-use images 808, 810, and 812 are frozen and displayed together with the classification results. Even in such a freeze display, the type of illumination light, the illumination mode, the recognition content, the classification result, and the like may be displayed as in FIGS. 11A to 11C.

The repeat control unit 204F (repeat control unit) repeats the above-described processes from Step S100 to Step S110 (Step S114) at a predetermined frame rate until the end condition is satisfied (during NO in Step S116) (repeat control step). The repeat control unit 204F can determine that, for example, in a case where there is an end instruction operation via the operation unit 208 or the imaging button 144, “the process ends” in the case where the acquisition of the image is completed.

In the endoscope system according to the first embodiment, by the above-described processing (determination, recognition, and display), it is not necessary for the user himself/herself to set the recognition content and display of the image according to the illumination mode, and the operation burden on the user can be thus reduced.

<Switching of Recognition and Display Due to Switching of Illumination Mode>

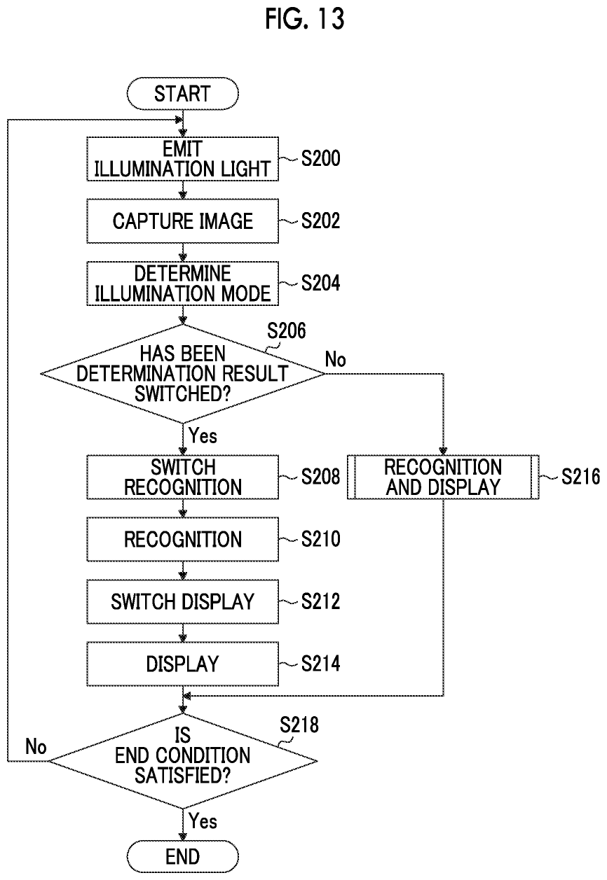

In the endoscope system 10, recognition and display can be switched according to the switching of the illumination mode while acquiring medical-use images in time series. For example, as shown in the flowchart of FIG. 13, the determination unit 204B determines whether or not the determination result has been switched (from the first illumination mode to the second illumination mode, or vice versa) (Step S206: determination step), and in a case where determination result has been switched (YES in Step S206), the recognition unit 204C switches between the first recognition and the second recognition according to the determination result being switched between the first illumination mode and the second illumination mode (Step S208: recognition step). Specifically, the CNN used for recognition is switched between the first CNN 214 (first recognizer) and the second CNN 215 (second recognizer). The recognition unit 204C performs recognition using the CNN after switching (Step S210: recognition step), and the display control unit 204D switches between the first display and the second display according to the switching between the first recognition and the second recognition (Step S212: display control step), and causes the monitor 400 (display device) to display the recognition result (Step S214: display control step). The first display and the second display can be performed in the same manner as in FIGS. 9A to 12. On the other hand, in a case where determination result has not been switched (NO in Step S206), recognition and display are performed in the same manner as in steps S106 to S114 of FIG. 8 (Step S216: recognition step, display control step).

The repeat control unit 204F repeats the above-described processes from Step S200 to Step S214 (Step S216) at a predetermined frame rate until the end condition is satisfied (during NO in Step S218) (repeat control step). Note that steps S200, S202, and S204 of FIG. 13 can be performed in the same manner as steps S100. S102, and S104 of FIG. 8, respectively. According to such processing, the user does not need to switch the recognition and display according to the switching of the illumination mode, and the operation burden can be thus reduced by reflecting the user's intention of “which recognition and display are to be performed”.

<Subsequent Processing of Recognition and Display>

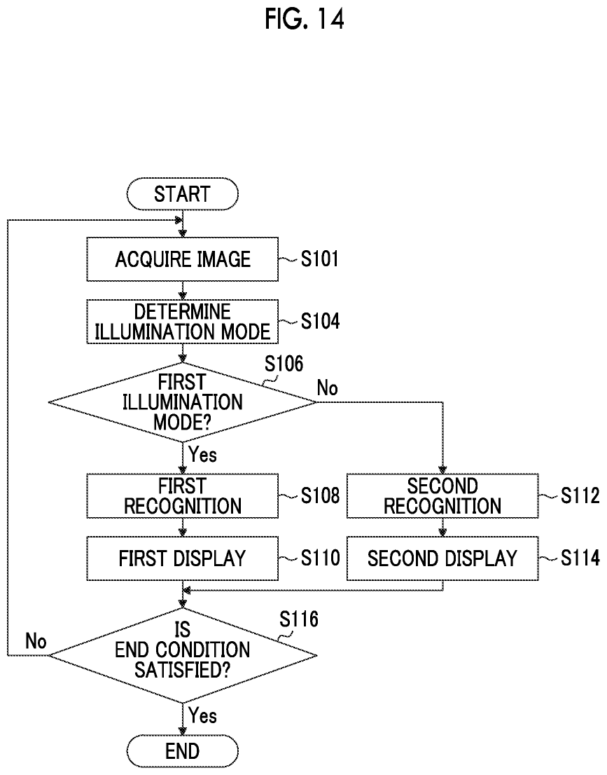

In the above-described embodiment, the mode in which the medical-use image is captured, recognized and displayed in parallel (refer to FIG. 8 and the like) has been described, but in the endoscope system 10, the image captured and recorded in advance can also be subsequently processed (determination, recognition, and display of the illumination mode). For example, the endoscope system 10 can recognize and display each frame of the endoscopic image (medical-use image) recorded in the recording unit 207 by the procedure shown in the flowchart of FIG. 14. In FIG. 14, the illumination mode of the image acquired in Step S101 (image acquisition step) is determined in Step S104. In a case where a setting history or the like of the illumination mode is recorded at the time of imaging, the determination unit 204B can determine the illumination mode by using the recorded information, and in a case where such information is not recorded, the determination unit 204B can analyze and determine the image by using the illumination mode determination CNN 213, the analysis unit 219, or the like. In the flowchart of FIG. 14, the same step numbers are assigned to the same processes as those of the flowchart of FIG. 8, and detailed description thereof will be omitted.

Such processing may be performed by a computer or a medical-use image processing device (a device independent of the endoscope system 10) that does not comprise an imaging portion (endoscope, light source device, imaging unit, and the like). In the case where processing is performed by such a medical-use image processing device or a computer, since it may not be able to directly acquire information on the illumination mode from the imaging portion, in that case, the determination unit may analyze the above-mentioned “information displayed on the display device together with the medical-use image” to make determination.

<Other Configuration Examples of Light Source>

Other configuration examples of the light source in the endoscope system 10 will be described. Even in the light sources having the configurations shown in these examples, the processing (determination, recognition, and display of the illumination mode) of the medical-use image processing method can be performed in the same manner as in the above-described embodiment.

### Example 1

As shown in FIG. 15, a light source device 320 (light source device) comprises a white light laser light source 312 (white light laser light source) that emits a white light laser as excitation light, and a phosphor 314 (phosphor) that emits white light (normal light) as first illumination light when irradiated with the white light laser, and a narrow-band light laser light source 316 (narrow-band light laser light source) that emits narrow-band light (an example of special light; for example, blue narrow-band light can be used, but green narrow-band light and red narrow-band light may also be used) as second illumination light. The light source device 320 is controlled by the light source control unit 350. In FIG. 15, the components other than the light source device 320 and the light source control unit 350 among the components of the endoscope system 10 are not shown.

### Example 2

As shown in FIG. 16, a light source device 322 (light source device) comprises a white light source 318 (white light source) that emits white light, a rotation filter 360 (white light filter, narrow-band light filter) in which a white light region that transmits the white light (normal light; first illumination light) and a narrow-band light region that transmits narrow-band light (an example of special light; second illumination light) are formed, and a rotation filter control unit 363 (first filter switching control unit) that controls the rotation of the rotation filter 360 to insert the white light region or the narrow-band light region into an optical path of the white light. The white light source 318 and the rotation filter control unit 363 are controlled by the light source control unit 350. In FIG. 16, the components other than the light source device 322 and the light source control unit 350 among the components of the endoscope system 10 are not shown.

In Example 2, as the white light source 318, a white light source that emits a wide band of light may be used, or white light may be generated by simultaneously emitting a light source that emits red, green, blue, and violet light. Further, the rotation filter 360 and the rotation filter control unit 363 may be provided in the light source 310 shown in FIG. 2.

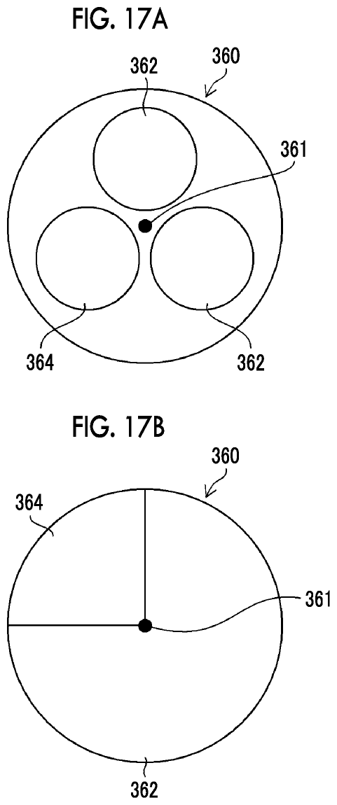

FIGS. 17A and 17B are diagrams showing an example of the rotation filter 360. In the example shown in FIG. 17A, the rotation filter 360 is formed with two circular white light regions 362 (white light filter) that transmit white light and one circular narrow-band light region 364 (narrow-band light filter) that transmits narrow-band light, and the white light region 362 or the narrow-band light region 364 is inserted into the optical path of the white light by rotating around a rotation shaft 361 under the control of the rotation filter control unit 363 (first filter switching control unit), whereby the subject is irradiated with the white light (first illumination light) or the narrow-band light (second illumination light). The narrow-band light region 364 can be a region that transmits any narrow-band light such as red, blue, green, and violet. Further, the number, shape, and arrangement of the white light region 362 and the narrow-band light region 364 are not limited to the example shown in FIG. 17A, and may be changed according to the irradiation ratio of the white light and the narrow-band light.

The shapes of the white light region and the narrow-band light region are not limited to the circular shape as shown in FIG. 17A, and may be fan-shaped as shown in FIG. 17B. FIG. 17B shows an example in which three-quarters of the rotation filter 360 is the white light region 362 and one-quarter is the narrow-band light region 364. The area of the fan shape can be changed according to the irradiation ratio of the white light and the narrow-band light. In the examples of FIGS. 17A and 17B, a plurality of narrow-band light regions corresponding to different narrow-band light beams may be provided in the rotation filter 360.

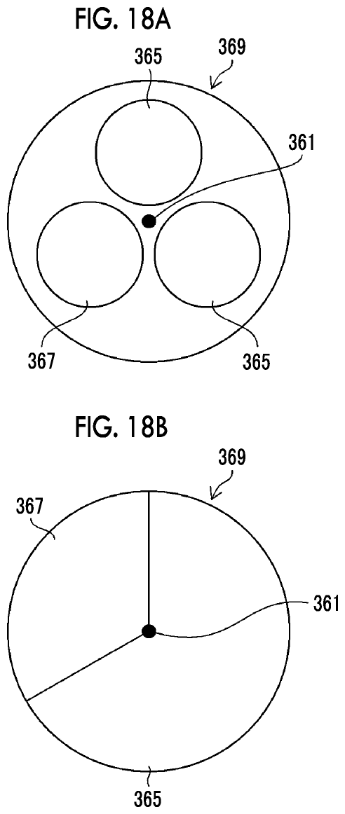

FIGS. 18A and 18B are diagrams showing another example of the rotation filter. As the white light source for the rotation filter shown in FIGS. 18A and 18B, the white light source 318 can be used as in the light source device 322 shown in FIG. 16. Further, unlike the rotation filter 360 shown in FIGS. 17A and 17B, a rotation filter 369 shown in FIG. 18A is not provided with a white light region that transmits white light, and is provided with two circular first narrow-band light regions 365 (first narrow-band light filter) that transmit a component of first narrow-band light (first special light; first illumination light) and one circular second narrow-band light region 367 (second narrow-band light filter) that transmits a component of second narrow-band light (second special light; second illumination light), of the white light. By rotating the rotation filter 369 around the rotation shaft 361 by the rotation filter control unit 363 (refer to FIG. 16; second filter switching control unit), the first narrow-band light region 365 (first narrow-band light filter) or the second narrow-band light region 367 (second narrow-band light filter) is inserted into the optical path of the white light emitted by the white light source 318, and the subject can be irradiated with the first narrow-band light or the second narrow-band light.

The shapes of the first narrow-band light region 365 and the second narrow-band light region 367 are not limited to the circular shape as shown in FIG. 17A, and may be fan-shaped as shown in FIG. 17B. FIG. 17B shows an example in which two-thirds of the rotation filter 369 is the first narrow-band light region 365 and one-third is the second narrow-band light region 367. The area of the fan shape can be changed according to the irradiation ratio of the first narrow-band light and the second narrow-band light. In the examples of FIGS. 17A and 17B, three or more types of narrow-band light regions corresponding to different narrow-band light beams may be provided in the rotation filter 369.

(Additional remark)

In addition to each aspect of the above-described embodiment, configurations to be described below are also included in the scope of the present invention.

(Additional Remark 1)

A medical image processing device comprising: a medical image analysis processing unit that detects a region of interest, which is a region to be noticed, on the basis of a feature quantity of pixels of a medical image; and a medical image analysis result acquisition unit that acquires an analysis result of the medical image analysis processing unit.

(Additional Remark 2)

In the medical image processing device, the medical image analysis processing unit detects presence or absence of a target to be noticed, on the basis of a feature quantity of pixels of a medical image, and the medical image analysis result acquisition unit acquires an analysis result of the medical image analysis processing unit.

(Additional Remark 3)

In the medical image processing device, the medical image analysis result acquisition unit acquires an analysis result of the medical image from a recording device, and the analysis result includes any one or both of the region of interest, which is a region to be noticed, included in the medical image and the presence or absence of the target to be noticed.

(Additional Remark 4)

In the medical image processing device, the medical image is a normal light image obtained by emitting light in a white range or light in a plurality of wavelength ranges as the light in the white range.

(Additional Remark 5)

In the medical image processing device, the medical image is an image obtained by emitting light in a specific wavelength range, and the specific wavelength range is a range narrower than a white wavelength range.

(Additional Remark 6)

In the medical image processing device, the specific wavelength range is a blue range or a green range of a visible range.

(Additional Remark 7)

In the medical image processing device, the specific wavelength range includes a wavelength range of 390 nm to 450 nm or 530 nm to 550 nm, and light in the specific wavelength range has a peak wavelength in a wavelength range of 390 nm to 450 nm or 530 nm to 550 nm.

(Additional Remark 8)

In the medical image processing device, the specific wavelength range is a red range of a visible range.

(Additional Remark 9)

In the medical image processing device, the specific wavelength range includes a wavelength range of 585 nm to 615 nm or 610 nm to 730 nm, and light in the specific wavelength range has a peak wavelength in a wavelength range of 585 nm to 615 nm or 610 nm to 730 nm.

(Additional Remark 10)

In the medical image processing device, the specific wavelength range includes a wavelength range where a light absorption coefficient in oxygenated hemoglobin is different from that in reduced hemoglobin, and light in the specific wavelength range has a peak wavelength in a wavelength range where a light absorption coefficient in oxygenated hemoglobin is different from that in reduced hemoglobin.

(Additional Remark 11)

In the medical image processing device, the specific wavelength range includes a wavelength range of 400±10 nm, 440±10 nm, 470±10 nm, or 600 nm to 750 nm, and light in the specific wavelength range has a peak wavelength in a wavelength range of 400±10 nm, 440±10 nm, 470±10 nm, or 600 nm to 750 nm.

(Additional Remark 12)

In the medical image processing device, the medical image is an in-vivo image that the inside of a living body is captured, and the in-vivo image has information on fluorescence emitted by a fluorescent material in the living body.

(Additional Remark 13)

In the medical image processing device, the fluorescence is obtained by irradiating the inside of the living body with excitation light which has a peak in a range of 390 nm to 470 nm.

(Additional Remark 14)

In the medical image processing device, the medical image is an in-vivo image that the inside of a living body is captured, and the specific wavelength range is an infrared wavelength range.

(Additional Remark 15)

In the medical image processing device, the specific wavelength range includes a wavelength range of 790 nm to 820 nm or 905 nm to 970 nm and light in the specific wavelength range has a peak wavelength in a wavelength range of 790 nm to 820 nm or 905 nm to 970 nm.

(Additional Remark 16)

In the medical image processing device, the medical image acquisition unit comprises the special light image acquisition unit that acquires a special light image having information on a specific wavelength range on the basis of a normal light image obtained by emitting light in a white range or light in a plurality of wavelength ranges as the light in a white range, and the medical image is a special light image.

(Additional Remark 17)

In the medical image processing device, a signal of the specific wavelength range is obtained by calculation based on color information of RGB or CMY included in the normal light image.

(Additional Remark 18)

The medical image processing device further comprising: a feature quantity image generation unit that generates a feature quantity image by calculation based on at least one of a normal light image obtained by emitting light in a white range or light in a plurality of wavelength ranges as the light in the white range or a special light image obtained by emitting light in a specific wavelength range, in which the medical image is a feature quantity image.

(Additional Remark 19)

An endoscope device comprising: the medical image processing device according to any one of Additional remarks 1 to 18; and an endoscope that acquires an image obtained by emitting at least one of light in a white wavelength range or light in a specific wavelength range.

(Additional Remark 20)

A diagnosis support device comprising: the medical image processing device according to any one of Additional remarks 1 to 18.

(Additional Remark 21)

A medical service support device comprising: the medical image processing device according to any one of Additional remarks 1 to 18.

Although the embodiments and other aspects of the present invention have been described above, the present invention is not limited to the above-described aspects, and various modifications can be made without departing from the spirit of the present invention.

### EXPLANATION OF REFERENCES

10: endoscope system
100: endoscope main body
102: hand operation part
104: insertion part
106: universal cable
108: light guide connector
112: flexible portion
114: bendable portion
116: distal end rigid portion
116A: distal end side end surface
123: illumination part
123A: illuminating lens
123B: illuminating lens
126: forceps port
130: imaging optical system
132: imaging lens
134: imaging element
136: drive circuit
138: AFE
141: air-supply and water-supply button
142: suction button
143: function button
144: imaging button
170: light guide
200: processor
202: image input controller
204: image processing unit
204A: image acquisition unit
204B: determination unit
204C: recognition unit
204D: display control unit
204E: reception unit
204F: repeat control unit
205: communication control unit
206: video output unit
207: recording unit
208: operation unit
209: sound processing unit
209A: speaker
209B: microphone
210: CPU
211: ROM
212: RAM
213: illumination mode determination CNN
214 first CNN
214A: input layer
214B: interlayer
214C: output layer
215: second CNN
216: convolution layer
217: pooling layer
218: fully connected layer
219: analysis unit
300: light source device
310: light source
310B: blue light source
310G: green light source
310R: red light source
310V: violet light source
312: white light laser light source
314: phosphor
316: narrow-band light laser light source
318: white light source
320: light source device
322: light source device
330: stop
340: condensing lens
350: light source control unit
360: rotation filter
361: rotation shaft
362: white light region
363: rotation filter control unit
364: narrow-band light region
365: first narrow-band light region
367: second narrow-band light region
369: rotation filter
400: monitor
800: medical-use image
801: region of interest
802: medical-use image
806: medical-use image
806A: frame
806B: marker
806C: marker
808: medical-use image
810: medical-use image
812: medical-use image
820: frame
830: region
840: region
842: region
S100 to S218: each step of medical-use image processing method

What is claimed is:

1. A medical-use image processing device comprising:
an image acquisition unit that acquires a medical-use image;
a determination unit that determines an illumination mode at the time when the medical-use image is captured;
a recognition unit that performs a first recognition for the medical-use image in a case where determination is made that the illumination mode is a first illumination mode, and performs a second recognition for the medical-use image in a case where determination is made that the illumination mode is a second illumination mode; and
a display control unit that causes a display device to display a first display according to a result of the first recognition in a case where the determination is made that the illumination mode is the first illumination mode, and causes the display device to display a second display according to a result of the second recognition in a case where the determination is made that the illumination mode is the second illumination mode.

2. The medical-use image processing device according to claim 1, wherein
the image acquisition unit acquires the medical-use image in time series,
the determination unit makes the determination on a frame constituting the medical-use image acquired in the time series,
the recognition unit switches between the first recognition and the second recognition according to a result of the determination being switched between the first illumination mode and the second illumination mode, and
the display control unit switches between the first display and the second display according to switching between the first recognition and the second recognition.

3. The medical-use image processing device according to claim 1, wherein the recognition unit detects a region of interest reflected in the medical-use image in the first recognition, and classifies the medical-use image in the second recognition.

4. The medical-use image processing device according to claim 3, wherein the recognition unit performs classification on the region of interest detected in the first recognition, in the second recognition.

5. The medical-use image processing device according to claim 3, wherein the display control unit causes the display device to display information indicating a detection position of the region of interest reflected in the medical-use image in the first display, and causes the display device to display information indicating a classification result of the medical-use image in the second display.

6. The medical-use image processing device according to claim 3, wherein the recognition unit includes:
a first recognizer that is constructed by learning and performs the first recognition, the first recognizer detecting the region of interest from the medical-use image; and
a second recognizer that is constructed by learning and performs the second recognition, the second recognizer classifying the medical-use image.

7. The medical-use image processing device according to claim 6, wherein the first recognizer and the second recognizer have a hierarchical network structure.

8. The medical-use image processing device according to claim 1, further comprising: a reception unit that receives an operation of a user,
wherein the determination unit makes the determination based on the received operation.

9. The medical-use image processing device according to claim 1, wherein the determination unit analyzes the acquired medical-use image to make the determination.

10. The medical-use image processing device according to claim 9, wherein the determination unit performs the analysis based on a distribution of color components in the medical-use image.

11. The medical-use image processing device according to claim 9, wherein the determination unit performs the analysis using a convolutional neural network.

12. The medical-use image processing device according to claim 1, wherein the determination unit analyzes information displayed on the display device together with the medical-use image to make the determination.

13. An endoscope system comprising:
the medical-use image processing device according to claim 1;
the display device;
an endoscope including an insertion part to be inserted into a test object and a hand operation part connected to a proximal end of the insertion part, the insertion part including a distal end rigid portion, a bendable portion connected to a proximal end of the distal end rigid portion, and a flexible portion connected to a proximal end of the bendable portion:
a light source device having the first illumination mode and the second illumination mode, the light source device irradiating the test object with first illumination light in the first illumination mode, and irradiating the test object with second illumination light in the second illumination mode; and
an imaging unit including an imaging lens that forms an optical image of the test object, and an imaging element on which the optical image is formed by the imaging lens.

14. The endoscope system according to claim 13, wherein the light source device irradiates the test object with normal light as the first illumination light and irradiates the test object with special light as the second illumination light.

15. The endoscope system according to claim 14, wherein the light source device comprises:
a white light laser light source that emits a white light laser as excitation light;
a phosphor that emits white light as the normal light when irradiated with the white light laser; and
a narrow-band light laser light source that emits narrow-band light as the special light.

16. The endoscope system according to claim 14, wherein the light source device comprises:
a white light source that emits white light as the normal light;
a white light filter that transmits the white light;
a narrow-band light filter that transmits a component of narrow-band light, as the special light, of the white light; and
a first filter switching control unit that inserts the white light filter or the narrow-band light filter into an optical path of the white light emitted by the white light source.

17. The endoscope system according to claim 13, wherein the light source device irradiates the test object with first special light as the first illumination light and irradiates the test object with second special light different from the first special light as the second illumination light.

18. The endoscope system according to claim 17, wherein the light source device comprises:
a white light source that emits white light;
a first narrow-band light filter that transmits a component of first narrow-band light, as the first special light, of the white light;
a second narrow-band light filter that transmits a component of second narrow-band light, as the second special light, of the white light; and
a second filter switching control unit that inserts the first narrow-band light filter or the second narrow-band light filter into an optical path of the white light emitted by the white light source.

19. A medical-use image processing method comprising:
an image acquisition step of acquiring a medical-use image;
a determination step of determining an illumination mode at the time when the medical-use image is captured;
a recognition step of performing a first recognition for the medical-use image in a case where determination is made that the illumination mode is a first illumination mode, and performing a second recognition for the medical-use image in a case where determination is made that the illumination mode is a second illumination mode; and
a display control step of causing a display device to display a first display according to a result of the first recognition in a case where the determination is made that the illumination mode is the first illumination mode, and causing the display device to display a second display according to a result of the second recognition in a case where the determination is made that the illumination mode is the second illumination mode.

20. The medical-use image processing method according to claim 19, wherein
the image acquisition step includes acquiring the medical-use image in time series,
the determination step includes making the determination on a frame constituting the medical-use image acquired in the time series,
the recognition step includes switching between the first recognition and the second recognition according to a result of the determination being switched between the first illumination mode and the second illumination mode, and
the display control step includes switching between the first display and the second display according to switching between the first recognition and the second recognition.
